(19) United States
(12) Patent Application Publication
(10) Pub. No.: US 2011/0244057 A1
(43) Pub. Date: Oct. 6, 2011

(54) COMBINATION THERAPIES WITH TOPIRAMATE FOR SEIZURES, RESTLESS LEGS SYNDROME, AND OTHER NEUROLOGICAL CONDITIONS

(76) Inventor: Bruce L. Ehrenberg, New York, NY (US)

(21) Appl. No.: 13/120,933

(22) PCT Filed: Sep. 25, 2009
(86) PCT No.: PCT/US2009/058495

§ 371 Date: Jun. 9, 2011

Related U.S. Application Data
(60) Provisional application No. 61/100,232, filed on Sep. 25, 2008.

(51) Int. Cl. A61K 33/26; A61K 31/35; A61K 33/06; A61P 25/00; A61P 25/28; A61P 25/08

(52) U.S. Cl. 424/648; 514/454; 424/682; 424/646

(57) ABSTRACT

The invention provides compositions and methods of treating various conditions, including neurological conditions, with pharmaceutical compositions including a sulfamate (e.g., topiramate) and magnesium.

### CROSS-REFERENCE TO RELATED APPLICATIONS

This application claims the benefit of the priority date of U.S. Application No. 61/100,232, filed Sep. 25, 2008. For the purpose of any U.S. patent that may issue from the U.S. national phase correlate of the present application, the content of the priority document is hereby incorporated by reference in its entirety.

### TECHNICAL FIELD

The present invention includes treatments for several neurological conditions, including epileptic seizures triggered by migraine with or without aura, eclamptic seizures and posterior encephalopathy of late pregnancy, seizures and status epilepticus associated with reversible posterior leucoencephalopathy in patients on immunosuppressants, Restless Leg Syndrome (RLS), and Periodic Limb Movement Disorder (PLMD), with various combinations of the agents topiramate (or another sulfamate), magnesium, and iron.

### SUMMARY

The present invention is based, in part, on the recognition that patients under physiologic stress from direct physical or mental trauma, often compounded by severe sleep deprivation (thus preventing repletion of the body's main energy source—ATP) may accumulate subtle tissue damage, especially in the posterior regions of the brain where the highest rate of routine waking metabolic activity is found. Those suffering from the movement disorders Restless Leg Syndrome (RLS) and Periodic Limb Movement Disorder (PLMD), patients suffering from a sleep disorder such as narcolepsy, and pregnant patients who are at risk of an eclamptic seizure can be treated with a sulfamate conforming to Formula I (e.g., topiramate) or with a combination of agents that include a compound of Formula I, magnesium (magnesium sulfate is currently the treatment of choice for eclampsia) and, optionally, iron. Accordingly, the invention features pharmaceutical compositions that include (a) a compound of Formula I or a pharmaceutically acceptable derivative thereof:

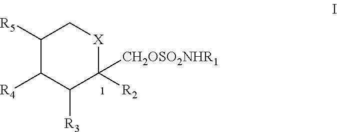

where X is CH₂ or oxygen, R₁ is hydrogen or lower alkyl, and R₂, R₃, R₄ and R₅ are independently hydrogen or lower alkyl, or R₂ and R₃ and/or R₄ and R₅ together may be a group of the following Formula II:

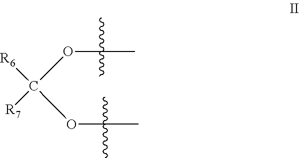

wherein R₆ and R₇ are the same or different and are hydrogen, lower alkyl or are alkyl and are joined to form a cyclopentyl or cyclohexyl ring, and (b) magnesium. While the invention is not limited to compositions that act by any particular mechanism, the amount of the magnesium can be sufficient to potentiate the effect of the compound of Formula I or the pharmaceutically acceptable derivative thereof. In some embodiments, the pharmaceutical compositions can include a therapeutically effective amount of a compound of Formula I.

In some embodiments R₂, R₃, R₄ and R₅ are independently hydrogen or lower alkyl. In other, separate embodiments, R₂ and R₃ and/or R₄ and R₅ together are a group of Formula II. In other words, in Formula I, R₂, R₃, R₄ and R₅ can be, independently, hydrogen or lower alkyl or, in Formula I, R₂ and R₃ and/or R₄ and R₅ together can be a group of Formula II.

Where X is CH₂, R₄ and R₅ can be alkene groups joined to form a benzene ring.

Where X is oxygen, R₂ and R₃ and/or R₄ and R₅ together can be a methylenedioxy group of Formula II:

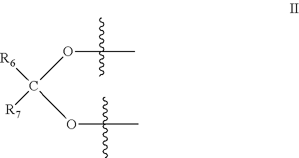

With respect to Formula II, R₆ and R₇ can be the same or different and can be hydrogen, lower alkyl or alkyl and can be joined to form a cyclopentyl or cyclohexyl ring.

In particular embodiments, the pharmaceutical composition of Formula I can be:
2,3-O-(1-methylethylidene)-4,5-O-sulfonyl-β-D-fructopyranose sulfamate;
2,3-O-(1-methylethylidene)-4,5-O-sulfonyl-β-L-fructopyranose sulfamate;
2,3-O-(1-methylethylidene)-4,5-O-sulfonyl-β-D-fructopyranose methylsulfamate;
2,3-O-(1-methylethylidene)-4,5-O-sulfonyl-β-D-fructopyranose butylsulfamate;
2,3-O-(1-methylethylidene)-4,5-O-sulfonyl-β-D-fructopyranose ethylsulfamate;
2,3-O-(1-methylethylidene)-4,5-O-sulfonyl-β-D-fructopyranose octylsulfamate;
2,3-O-(1-methylethylidene)-4,5-O-sulfonyl-β-D-fructopyranose 2-propenylsulfamate;
2,3-O-(1-methylethylidene)-4,5-O-sulfonyl-β-D-fructopyranose phenylmethylsulfamate;
2,3-O-(1-methylethylidene)-4,5-O-sulfonyl-β-D-fructopyranose cyclopropylsulfamate;
2,3-O-(1-methylethylidene)-4,5-O-sulfonyl-β-D-fructopyranose cyclobutylsulfamate;
2,3-O-(1-methylethylidene)-4,5-O-sulfonyl-β-D-fructopyranose (2,2,2-trifluoroethyl)sulfamate;
2,3-O-(1-methylethylidene)-4,5-O-sulfonyl-β-D-fructopyranose dimethylsulfamate;
2,3-O-(1-methylethylidene)-4,5-O-sulfonyl-β-D-fructopyranose diethylsulfamate;
2,3-O-(1-methylethylidene)-4,5-O-sulfonyl-β-D-fructopyranose azido sulfamate;
(S)-2,3-O-(1-methylethylidene)-4,5-O-sulfinyl-β-D-fructopyranose sulfamate;
(R)-2,3-O-(1-methylethylidene)-4,5-O-sulfinyl-β-D-fructopyranose sulfamate;
2,3-O-(1-ethylpropylidene)-4,5-O-sulfonyl-β-D-fructopyranose sulfamate;
2,3-O-(1-methylethylidene)-4,5-O—[N-(4-methylbenzenesulfonyl)imidosulfonyl]-β-D-fructopyranose sulfamate;
2,3-O-(1-methylethylidene)-4,5-O—[N-(4-methylbenzenesulfonyl)imidosulfonyl]-β-D-fructopyranose sulfamate;
2,3-O-(cyclohexylidene)-4,5-O-sulfonyl-β-D-fructopyranose sulfamate; or
(S)-4,5-O—[N-(1,1-dimethylethoxycarbonyl)imidosulfinyl]-2,3-O-(1-methylethy lidene)-beta-D-fructopyranose sulfamate.

In any of the present embodiments, the compound of Formula I can be 2,3:4,5-bis-O-(1-methylethylidene)-beta-D-fructopyranose sulfamate (topiramate).

While pharmaceutically acceptable derivatives are discussed further below, we note here that such derivatives encompass pharmaceutically acceptable salts (e.g., an alkali metal salt, an ammonium salt, or a crystalline choline salt). Pharmaceutically acceptable derivatives can also be compounds of Formula I that include a masking chemical group (e.g., an imidate group) that is dissociated from the compound in vivo.

The precise amounts of the agents administered can vary and can be readily determined by one of ordinary skill in the art using the techniques and procedures known to one of ordinary skill in the art of drug discovery. Generally, the agents of the present composition (e.g., a compound of Formula I and magnesium) can be used in amounts at or below the minimum effective dosage needed for either compound given separately. Generally, it is expected that the sulfamates can be effectively administered at levels lower than the levels at which they are currently, typically prescribed. For example, a compound of Formula I can be used as described herein when present in a pharmaceutical composition that contains magnesium at a unit dosage of about 15-75 mg (e.g., 25-50 mg). Magnesium can be present at a unit dosage of 150-400 mg (e.g., about 250 mg of the oxide). However, it is common to greatly escalate the dosage when treating intractable eclampsia, sometimes to as high as 6 grams of the sulfate in a bolus or rapidly infused intravenous push.

The pharmaceutical compositions can further include iron. For example, the compositions can include about 5 to 30 mg of elemental iron, which may be present in the form of iron (II) sulfate, ferrous gluconate, or NaFeEDTA. It is known that some formulations of iron, especially when ingested orally, may cause slowed gastrointestinal motility leading to constipation. To the contrary, orally ingested magnesium has a known laxative effect and thus, in the combined form, there is often a need to adjust the dosing of the two elements to reach a balance with respect to gastrointestinal motility. In some embodiments, the present compositions can include only magnesium and iron to produce satisfactory improvement with minimal or no side effects.

Hypomagnesemia, hypercalciuria, and nephrocalcinosis (HHN) are the most common causes of progressive nephropathy that presents with low Mg²⁺ and for which early replacement of the mineral ion can delay or prevent kidney damage. Thus, adding Mg²⁺ to the Formula I composition could circumvent the self-contained carbonic anhydrase inhibitory activity and avoid the markedly elevated renal stone production rate that destroys the kidney. Since treatment of the susceptible population may prevent the bad outcome, it could be justified to consider recommending to the public the increased use of the over-the-counter mineral as a supplement if the data showing widespread magnesium deficiency can be verified. Magnesium is a mineral like fluoride and chloride, two ions that have already been routinely added to the general water supply.

The present pharmaceutical compositions can be formulated for oral or parenteral administration, and particular formulations are described further below.

The compositions, including those that contain a compound of Formula I (with or without a potentiating amount of magnesium) are also useful in treating a patient who is suffering from RLS or PLMD, especially if that patient is already taking pramipexole, known to cause a severe insomnia of its own, associated with profound daytime sleepiness. Such severe sleep disruption can increase the risk of a true epileptic seizure, or exacerbate a pre-existing seizure disorder, especially in genetically pre-disposed individuals who have inherited one of the known epilepsy mutations (e.g., a chloride or potassium channelopathy). In addition, familial hemiplegic migraine is a relatively rare syndrome which presents with atypical seizures involving unilateral slow rhythmic 1 Hz twitching, poorly responsive to routine anticonvulsants, and associated with PLEDs in the EEG. These patients have a known channelopathy involving the calcium pores, with the majority of families showing linkage to the CACN2 gene, where a variety of mutations have been found. The same applies to major loss of sleep in patients suffering from migraine with aura, with attendant memory disturbance or dizziness/lightheadedness superimposed on the more typical symptoms of hemianopsia, hemiplegia, or aphasia.

Since both migraine and RLS/PLMD have an almost 2:1 female predominance, even greater in the third decade when menstruation in women is fully expressed, iron-deficiency anemia frequently co-exists. However, the mechanism here is more complex, with the sleep-deprivation developing gradually, through the effect of menstruation-related anemia on respiratory function, which in turn causes sleep disruption when the hematocrit and the SaO₂ (as measured by oximeter) drop below a critical comfort level for sleep and breathing. Third trimester physical discomfort during sleep added to the impairment of breathing due to sleep-hypopnea or sleep-apnea can lead to hypertension of pregnancy or full-blown eclampsia.

Similarly, in treating a patient who is with at risk of experiencing a seizures due to pre-eclampsia or eclampsia of pregnancy, or posterior reversible encephalopathy in any setting, the ionized magnesium levels may be even lower than the current mildly-deficient population figures. It is increasingly noted in association with prolonged stress, or severe sleep-deprivation that in the absence of supplemental ingestion, the ionized levels can drop to a range where the seizure threshold is significantly lowered. It should also be noted that diuretic use can lead to magnesium depletion via renal wasting.

The methods can be carried out by (a) identifying a patient in need of treatment; and (b) administering to the patient a pharmaceutical composition described herein. As patients who have narcolepsy tend to have abundant PLMD—second only to RLS patients—and this may be a prime cause of the profound sleepiness in both syndromes, it stands to reason that, for narcolepsy, identifying a patient in need of treatment may include determining which of the syndrome's specific major symptoms are present, narcoleptic sleep attacks, and/or cataplexy (sudden daytime attack of generalized weakness often leading to a fall and attributed to aberrant REM sleep). Narcoleptic patients, regardless of any symptoms of PLMD, can also be treated with a sulfamate, including a sulfamate formulated as described herein (i.e., one formulated with or administered with magnesium and/or iron).

The present compositions can be used to treat a patient who has, or who is at risk of developing, any one or more of the conditions previously recognized as being treatable with a compound of Formula I (e.g., topiramate). The methods can be carried out by first identifying a patient who has a condition recognized as being treatable with a compound of Formula I (e.g., topiramate). Many of these conditions are described below. These and possibly others will be known to one of ordinary skill in the art. Once the patient has been identified as a candidate for treatment, one would administer a pharmaceutical composition as described herein.

Treatable conditions include neurological disorders, including those associated with seizure activity (e.g., a form of epilepsy). The neurological disorder can also be one characterized by migraine headache, migraine aura (alone or followed by migraine headache), cluster headache, or migraine with prolonged aura. The neurological condition being treated can thus include epileptic seizures triggered by migraine with or without aura. The seizures may also be eclamptic seizures, associated with posterior encephalopathy of late pregnancy, or seizures and status epilepticus associated with reversible posterior leucoencephalopathy in patients on immunosuppressants.

In additional treatable conditions, the patient is suffering from bipolar disorder, alcoholism, obesity, optionally associated with binge eating, periventricular leukomalacia, bulimia nervosa, obsessive-compulsive disorder, or idiopathic intracranial hypertension. The patient may also be addicted to nicotine or cocaine. Thus the compositions described herein (e.g., those including a sulfamate of Formula I and magnesium) can be included in a smoking cessation or anti-addiction program.

The present compositions can be administered as a component of a combination therapy. For example, for treating migraine, the present compositions can be administered together with another composition, several of which are currently known, that is suitable for treating migraine.

Other treatable conditions include: impulse control disorders (e.g., kleptomania), chronic neurodegenerative disease (e.g., Alzheimer's disease, vascular dementia, Parkinson's disease, Huntington's disease, multiple sclerosis, amyotrophic lateral sclerosis, or a depression, and post-traumatic stress disorder. The neurological disorder can be characterized by neuropathic pain, as in diabetic neuropathy. Patients experiencing abdominal or visceral pain can, however, also be treated.

The neurological deficit or paroxysmal event (e.g., seizure) can also result from head trauma or a spinal injury, and the treatments outlined herein may be useful during both the acute and convalescent stages of the illness (thereby reducing the number of new compounds introduced into the patient).

In other embodiments, the invention features the use of the components described herein (e.g., a compound of Formula I, such as topiramate, and magnesium) in the preparation of a medicament and use in the preparation of such medicaments for the treatment of one or more of the conditions described herein.

Other treatable conditions, including conditions that are unrelated to neurological disorders, are described further below, and other features, objects, and advantages of the invention will be apparent from the detailed description and from the claims. While the present methods are not so limited, it is believed they will, in at least some embodiments, enhance the efficacy and tolerability of treatment, relative to, for example, treatment with a sulfamate (e.g., topiramate) alone.

### DETAILED DESCRIPTION

The present invention encompasses compositions including a sulfamate (as herein defined), magnesium, and, optionally, iron as well as treatments using pharmaceutical preparations of those compositions to treat a variety of conditions. These conditions include stress-induced paroxysmal neurological events, including non-epileptic seizures triggered by migraine aura, eclampsia with posterior encephalopathy of late pregnancy, seizures associated with reversible posterior leucoencephalopathy in patients on immunosuppressants, and true epilepsy often exacerbated by remediable sleep-deprivation disorders such as sleep-apnea syndrome, restless legs syndrome (RLS) and periodic limb movements disorder (PLMD).

The present invention is based, in part, on the recognition that patients subjected to moderately severe physical or mental trauma may develop stress reactions with release of catecholamines and corticosteroids leading to glutamatergic and oxidative tissue destruction, and occasionally progress to permanent loss of clinical function typical of ischemic stroke due to arterial occlusion. With non-occlusive episodes of ischemia, it is rare to find brain infarction other than in cases of migrainous infarction or migraine with prolonged aura, wherein the onset of symptoms correlate with the appearance of cortical spreading depression of Leãu (CSD). The underlying mechanism of the latter phenomenon has been an enigma for over 70 years, but can be consistently observed when direct application of KCl to the cortex of a rat is followed by induction of localized changes in the EEG, blood flow, and cerebral metabolism. With the onset of migraine attacks in humans, it is thought that C SD occurs as a response to stress. It is also known that patients, under stress, develop insomnia and secondary sleep deprivation, as well as magnesium and iron depletion. The lack of normal restorative sleep impairs glucose metabolism and slows ATP repletion—both critical for brain function. In this setting there may be compilation of near-critical tissue dysfunction and even some briefly detectable clinical deficits, but without permanent neuronal damage, despite transiently imageable MRI changes. Peculiarly, these pathologic changes are skewed toward the posterior cerebral white matter (in close proximity to brain regions where baseline waking metabolic activity is highest). Possibly by way of a metabolic supply/demand mismatch, such conditions may progress to migraine with prolonged aura or eclamptic seizure and possibly to TIA (Transient Ischemic Attack), but not (acutely) to stroke.

Patients suffering severe stress or sleep-deprivation from disorders such as Restless Leg Syndrome (RLS), Periodic Limb Movement Disorder (PLMD), narcolepsy, eclampsia, and immunosuppressant induced seizures can be treated with a sulfamate conforming to Formula I (e.g., topiramate) or with a combination of agents that include a compound of Formula I, magnesium (magnesium sulfate is currently the treatment of choice for eclampsia) and, optionally, iron.

The present invention is based, in part, on the discovery that certain conditions, including conditions associated with seizures, RLS, PLMD (periodic limb movements during sleep), narcolepsy, and eclampsia, can be treated with a sulfamate of Formula I.

As noted, the present invention also features pharmaceutical compositions that include a compound of Formula I and a potentiating amount of magnesium. Potentiation occurs when one drug increases the effect of another. The potentiating agent (here, magnesium), may or may not have an effect of its own. The compositions can include, or can be administered together with, additional active agents, including iron, a vitamin, and/or a pharmaceutical agent previously recognized as being useful in the treatment of the specified condition. The inclusion of iron may be especially beneficial in treating conditions that are aggravated by an iron deficiency or anemia (e.g., RLS). Thus, the conditions provided above (RLS, PLMD, narcolepsy, pre-eclampsia, and eclampsia) can be treated with pharmaceutical compositions that include a compound of Formula I, with or without a potentiating amount of magnesium and/or iron, and conditions previously recognized as treatable with a compound of Formula I can be treated with a compound of Formula I and a potentiating amount of magnesium. These conditions include epilepsy, migraine, Lennox-Gastaut syndrome, bipolar disorder, alcoholism, obesity (especially as it relates to binge eating), post-traumatic stress disorder, periventricular leukomalacia in preterm infants after an hypoxic-ischemic injury, bulimia nervosa, obsessive-compulsive disorder, smoking, idiopathic intracranial hypertension, neuropathic pain, cluster headace and cocaine addiction.

While the present compositions are not limited to those that function by any particular mechanism, the inclusion or co-administration of magnesium can allow a compound of Formula I (e.g., topiramate) to provide effective relief at a dosage lower than the dosage that would be required in the absence of magnesium. Preferably, the therapeutic effect of magnesium (e.g., magnesium sulfate) is synergistic with the therapeutic effect of the compound of Formula I (e.g., topiramate). The “reverse” may also be true in that the compound of Formula I may allow one to administer a lower dose of magnesium to, for example, a patient who has pre-eclampsia or eclampsia, than would be effective in the absence of the compound of Formula I. The therapeutic effect of the two drugs in combination can be greater than the sum of the individual effects of the drugs when each drug is administered alone. This may be especially important when there is a high rate of ion flux in the glutamatergic NMDA-type calcium channel since magnesium can block this channel, thereby modulating the harmful oxidative effects of over-activating this excitatory pathway.

Pharmaceutical compositions: The present compositions can include magnesium and a compound of Formula I or a pharmaceutically acceptable derivative thereof. As noted, the compositions can also include additional active ingredients, such as iron, and may be formulated to include additional active agents useful in treating a given condition. For example, where a patient has sustained a head injury, the present compositions can include an analgesic or anti-inflammatory compound. For treating any of the conditions described herein, the active agents can be administered at the same time (e.g., in a common formulation, such as a pill) or at somewhat different times (e.g., sequentially within a matter of minutes or hours) and/or by the same or different routes of administration. For example, an iron tablet may be given as a supplement. Formulations and routes of administration are described further below.

A compound of Formula I suitable for use in the featured methods is a sulfamate of the following formula:

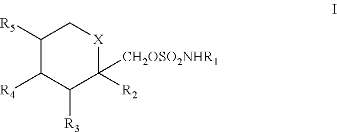

where X is CH₂ or oxygen; R₁ is hydrogen or lower (C₁-C₆) alkyl; and R₂, R₃, R₄ and R₅ are independently hydrogen or lower alkyl; and R₂, R₃, R₄ and R₅ are independently hydrogen or lower alkyl and/or R2 and R3 and/or R4 and R5 together may be a group of the following formula (II). When X is CH₂, R₄ and R₅ may be alkene groups joined to form a benzene ring, and when X is oxygen, R₂ and R₃ and/or R₄ and R₅ together may be a methylenedioxy group of Formula II:

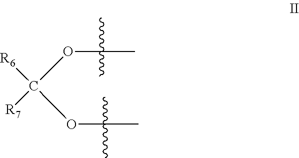

where R₆ and R₇ are the same or different and are hydrogen, lower alkyl or are alkyl and are joined to form a cyclopentyl or cyclohexyl ring.

The compositions can include various individual anomers, diastereomers and enantiomers of a compound of Formula I, as well as mixtures thereof.

In particular embodiments, R₁ can be hydrogen or an alkyl of about 1 to 4 carbons, such as methyl, ethyl and iso-propyl. The alkyl can be a straight or branched chain alkyl. The alkyl groups represented by R₂, R₃, R₄, R₅, R₆, and R₇ can be of about 1 to 3 carbons and include methyl, ethyl, iso-propyl and n-propyl. When X is CH₂, R₄ and R₅ may combine to form a benzene ring fused to the 6-membered X-containing ring (i.e., R₄ and R₅ can be defined by the alkatrienyl group ═CH—CH═CH—CH═).

For some compounds of Formula I, X is oxygen and both R₂ and R₃ and R₄ and R₅ together are methylenedioxy groups of Formula II, wherein R₆ and R₇ are both hydrogen, both alkyl, or combine to form a spiro cyclopentyl or cyclohexyl ring, in particular where R₆ and R₇ are both alkyl such as methyl. In a second group of compounds, X is CH₂, and R₄ and R₅ are joined to form a benzene ring. In a third group of compounds, both R₂ and R₃ are hydrogen.

Exemplary compounds of Formula I include tetrahydro-2H-pyran-2-yl)methane sulfamate; 2,3:4,5-bis-O-(1-methylethyldiene)-β-D-fructopyranose sulfamate; and 2,3:4,5-bis-O-(1-methylethyldiene)-β-D-fructopyranose methylsulfamate. A preferred compound is 2,3:4,5-bis-O-(1-methylethyldiene)-β-D-fructopyranose sulfamate, also known as topiramate. Topiramate has the chemical structure shown in Formula III:

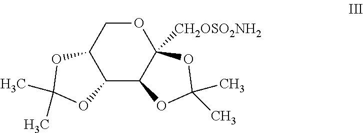

A “pharmaceutically acceptable derivative” is any pharmaceutically acceptable salt, ester, or salt of such ester of the compounds of Formula I or any other compounds which, upon administration to the patient, provides (directly or indirectly) a compound of Formula I or an active metabolite or residue thereof. Thus, the derivative can be a prodrug.

The pharmaceutically acceptable salt can be, for example, an alkali metal salt (e.g., sodium and potassium); an ammonium salt (e.g., a monoalkylammonium salt, a dialkylammonium salt, a trialkylammonium salt, or a tetraalkylammonium salt); or a tromethamine salt. For example, the pharmaceutically acceptable derivative can be a salt of topiramate.

Pharmaceutically acceptable salts of the compounds of Formula I include those derived from pharmaceutically acceptable, inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, formic, benzoic, malonic, napthalene-2-sulphonic and benzenesulphonic acids. Other acids such as oxalic acid, while not in themselves pharmaceutically acceptable, may be useful in the preparation of salts that are useful as intermediates in obtaining the present compounds and their pharmaceutically acceptable acid addition salts. Salts derived from appropriate bases include alkali metal (e.g., sodium), alkaline earth metal (e.g., magnesium), ammonium, and NR₄ (where R is C₁₋₄ alkyl) salts.

Other derivatives include a polymorph, solvate, dehydrate, or co-crystal of a compound of Formula I as described, for example, in U.S. Pat. No. 6,559,293 (the contents of which are incorporated by reference herein). Hydrates and other solvates of the compound of Formula I are included within the scope of the invention. The compounds of Formula I can also take the form of a crystalline choline salt, as described in U.S. Pat. No. 7,041,650 (the contents of which are incorporated by reference herein).

It will be appreciated by one of ordinary skill in the art that the pharmaceutically acceptable derivatives of a compound of Formula I may be derivatized at more than one position.

Other pharmaceutically acceptable derivatives are derivatives in which the sulfamate portion of the compound of Formula I is masked by a chemical group (e.g., an imidate group) that can be removed in a physiological milieu to generate the parent drug, as disclosed in U.S. Pat. No. 5,258,402 (the contents of which are incorporated by reference herein). The masked compound may be referred to as a pro-drug.

Other derivatives include sorbopyranose sulfamates (as described in U.S. Pat. No. 5,384,327), fructopyranose cyclic sulfites and sulfates (as described in U.S. Pat. No. 5,242,942), phenylethyl sulfamates (as described in U.S. Pat. No. 4,792,569), and acetazolamide (as described in U.S. Pat. Nos. 2,554,816 and 2,980,679).

While it is possible that, for use in the featured methods, compounds of Formula I may be administered as the raw chemical, it is preferable to present the active ingredient as a pharmaceutical formulation or composition which may further include a pharmaceutically acceptable carrier. The carrier must be “acceptable” in the sense of being compatible with the other ingredients of the formulation and not deleterious to the subject. Generally, the pharmaceutical compositions are non-toxic.

When magnesium is present in the composition or co-administered, it can be in the form of a salt, such as magnesium sulfate, magnesium sulfate heptahydrate, magnesium hydroxide, magnesium chloride, magnesium oxide, magnesium chloride hexahydrate, magnesium citrate, and/or trimagnesium dicitrate. Magnesium can also refer to elemental magnesium, for example, Mg(II) (e.g., Mg²⁺), including, but not limited to, instances in which the amount of elemental magnesium is expressed in moles or grams, even though the magnesium comprises a pharmaceutically acceptable salt thereof. For example, ˜120 gms of magnesium sulfate (e.g. MgSO₄) contains ˜24 gms of elemental magnesium.

When iron is present in the composition or co-administered, it can be in the form of a pharmaceutically acceptable iron salt. For example, the iron can be iron(II) sulfate, ferrous sulfate, ferrous sulfate heptahydrate, iron(II) gluconate, ferrous gluconate, amino-acid chelated iron(II), NaFeEDTA, iron(II) fumarate, iron(III) citrate, iron(II) succinate, iron(II) chloride, iron(II) glycine sulfate, iron(II) aspartate, sodium iron(III) gluconate, iron(III) hydroxide polymaltose complex, ferric sorbitol citrate complex, or iron(III) saccharate. The iron may be supplied in the form of iron-containing multivitamins. Iron can also refer to elemental iron, for example, Fe(II) (e.g., Fe²⁺), but as used herein, is not limited to instances wherein the amount of elemental iron is expressed in moles or grams, even though the iron comprises a pharmaceutically acceptable salt thereof. The term bioavailable iron broadly means iron-containing salts, chelates, and vitamins in which the iron is absorbable and utilizable by the subject.

Agents are “co-administered” when they are administered sequentially or simultaneously to the same patient. For example, the co-administered therapeutic agents can be part of the same formulation, or administered within 1, 2, 3, 4, 5, 6, 9, 12, 15, 18, 21, 24 or more hours of one another. Thus, co-administration encompasses the administration of two or more agents (e.g., therapeutic agents) at staggered times. One therapeutic agent might be administered more frequently than the other therapeutic agent or agents.

Commercially available pharmaceutical formulations of topiramate include synthetic iron oxide and magnesium stearate in such small quantities that the iron and magnesium are excipients (or “fillers” or “carriers”). The featured methods of co-administering topiramate, magnesium and, optionally, iron, contemplate dosing levels of magnesium and iron that are therapeutically effective in their own right (i.e., at levels where the effect is caused directly by the magnesium and/or iron) or by potentiating the effect of another agent (e.g., a compound of Formula I). The featured methods contemplate dosing levels of magnesium and/or iron that are in excess of the levels currently used, wherein magnesium and iron are included as excipients.

To prepare the pharmaceutical compositions of the invention, one or more sulfamate compounds of Formula I can be intimately admixed with magnesium, any optional agents (e.g., iron), and a pharmaceutical carrier according to conventional pharmaceutical compounding techniques. The carrier can take a wide variety of forms, depending on the form of the final preparation desired for administration (e.g., depending on whether delivery is contemplated by an oral or parenteral route of administration).

In preparing the compositions in oral dosage form, any of the usual pharmaceutical media can be employed. Thus, for liquid oral preparations (e.g., suspensions, elixirs and solutions), suitable carriers and additives include water, glycols, oils, alcohols, flavoring agents, preservatives, coloring agents and the like. For solid oral preparations (e.g., powders, capsules and tablets), suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. Aerosol sprays or capsules for absorption through the oral mucosa can be prepared as described in U.S. Pat. No. 6,977,070 (the contents of which are incorporated by reference herein).

Despite the advantages of oral formulations, the present compositions can also be prepared for intravenous, transdermal, subcutaneous, intraperitoneal, intrathecal, subdural, or intramuscular administration.

If desired, the compositions may be formulated as described in U.S. Pat. No. 6,569,463 (the contents of which are incorporated by reference herein), where the compositions take the form of a solid carrier that includes a substrate and an encapsulation coat. The coat includes an admixture of a therapeutically effective amount of an active ingredient (here, a compound of Formula I and/or magnesium), an effective solubilizing amount of at least one hydrophilic surfactant, and a lipophilic additive, which may be a lipophilic surfactant, a triglyceride, or a combination thereof.

If desired, the compositions may be formulated as an oil-in-water emulsion, as described in U.S. Pat. No. 6,720,001 (the contents of which are incorporated by reference herein).

Suppositories may be prepared, in which case cocoa butter could be used as the carrier. For other parenteral formulations, the carrier will usually comprise sterile water, although other ingredients that aid solubility or preserve shelf-life may be included.

Topiramate is currently available for oral administration in round tablets containing 25 mg, 100 mg or 200 mg of active agent. The tablets contain the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80. Any of these inactive ingredients may be included in the present compositions at levels that are non-toxic and do not elicit a physiologic response.

The present compositions can also be formulated for sustained release as described, for example, in U.S. Pat. No. 6,682,759 (which is incorporated by reference herein and which describes the manufacture of oral dosage forms delivering both immediate-release and sustained-release drugs).

For young patients or any patients who have difficulty swallowing medication, the present compositions can be formulated as described in U.S. Pat. No. 6,696,091 (the contents of which are incorporated by reference herein). In such formulations, the solid dosage formulation is a sprinkle formulation that includes core particles of the active agent taste-masked with a second layer. A compound of Formula I (e.g., topiramate) can be included in granular or crystalline form with one or more additional agents (e.g., magnesium) and one or more excipients, which are then formed into granules or beads by known techniques (e.g., roller compaction and comminution, extrusion-spheronization or other methods of forming granules or beads). The resulting microspheres may be sprinkled onto soft food and swallowed by the patient along with the food.

In some embodiments, the present compositions can also include a tramadol material as described in U.S. Pat. No. 6,562,865 (the content of which is incorporated by reference herein). For example, the compositions can include a tramadol material, magnesium, and a compound of Formula I, where the tramadol material and the compound of Formula I (e.g., topiramate) are present in a ratio based on a fraction of their respective ED₅₀ values. The ratio can be from about 1:1 to about 300:1 or from about 1:1 to about 1:300. As noted, the compositions, including those having a tramadol material, can also include iron or can be administered with an iron supplement. (To the extent that the tramadol material may have clinically detectable activity at the opioid receptors, it should be kept in mind that the opioids are known to have efficacy in treating RLS.)

Indications: The present compositions containing a compound of Formula I and an active and/or potentiating amount of magnesium are useful in the treatment of any condition for which a compound of Formula I is useful. This includes the treatment of neurological disorders, such as simple and complex partial seizure epilepsy with or without secondary generalized seizures, as epilepsy is treatable with topiramate (Topomax®).

Headaches, including cluster headaches and migraine, as well as migraine aura, can also be treated with topiramate and the compositions described herein. Migraine is typically a benign recurring headache and/or neurologic dysfunction. Classic migraine (migraine with aura) refers to the syndrome of a severe, throbbing headache which often is preceded by sensory, motor or visual symptoms, referred to as the “aura.” Common migraine denotes a headache without the aura and is the most frequent headache type reported by patients.

For the treatment of migraine, a compound of Formula I, magnesium and, optionally, iron, can be co-administered with an additional therapeutic agent for migraine treatment. Such agents include timolol, divalproex, gabapentin, propanolol, amitriptyline, verapamil, phenelzine, methysergide, aspirin, naproxen, ibuprofen, furosemide, furosemide-related compounds, loop diurectics, thiazides and thiazide-related compounds, phenyloin, carbamazepine, barbiturates, phenobarbital, pentobarbital, mephobarbital, trimethadione, mephenyloin, paramethadione, phenthenylate, phenacemide, metharbital, benzchlorpropamide, phensuximide, primidone, methsuximide, ethotoin, aminoglutethimide, diazepam, clonazepam, clorazepate, fosphenyloin, ethosuximide, valporate, felbamate, gabapentin, lamotrigine, vigrabatrin, tiagabine, zonisamide, clobazam, thiopental, midazoplam, propofol, levetiracetam, oxcarbazepine, CCPene, GYK152466, sumatriptan, mefenamic acid, flufenamic acid, tolfenamic acid, florinal, fioricet, frovatriptan, naratriptan, canesartan, lisinopril, atenolol, metoprolol, nadolol, fluoxetine, dihydroergotamine mesylate, DHE-25, riboflavin, coenzyme Q10, botulinum toxin type A, and chlorpromazine. The additional therapeutic can be administered before, at the same time as, or after treatment with magnesium and the compound of Formula I.

Where a subject suffers from cluster headaches, the present compositions may induce cluster remission or reduce cluster period duration. Terms such as “subject,” “patient,” and “individual” are used interchangeably and refer to a mammal amenable to treatment. While the present treatments are certainly intended for human patients, the invention is not so limited. Veterinary use is also contemplated and would include the treatment of domesticated animals, including those kept as pets and as livestock.

As noted in connection with cluster headaches, the treatments may vary in their outcome. While prevention or remission of symptoms is preferable, the invention is not so limited. The present methods are therapeutic where they reduce the risk that a patient will experience an adverse event (e.g., where they reduce the risk that a pre-eclamptic patient will suffer a seizure); where they delay the onset of a condition (e.g., where they delay the onset of diabetes); and/or where they reduce the frequency or severity of a symptom associated with the condition being treated. Thus, the terms “treat” and “treatment” refer to therapy in an amount, manner, and/or mode sufficient to improve or ameliorate a symptom of an existing disorder (by affecting the symptom and/or by affecting a cause of the disorder), or a parameter associated with a disorder, or which prevents or reduces progression of a disorder. The treatment can be characterized as promoting primary and secondary prophylaxis by preventing the development of a disease or a symptom, or, if the disease has already developed, by protecting the subject against deterioration, respectively.

For treating cluster headaches, and for treating certain other conditions described herein, particular dosages are provided to assist in the clinical perfection of the compositions. One of ordinary skill in the art will recognize, however, that appropriate doses will vary depending on a number of factors, any of which can be assessed in the clinical trial and/or drug development process. For example, a dosage may vary with the particular compound of Formula I used; with the forms of iron and magnesium selected; with the severity of the disease; the route of administration; and with parameters particular to a given patient (e.g., their age, weight, sex, tolerance of susceptibility to an active ingredient, and the like).

The present compositions can be made with therapeutically effective amounts of the agents described herein, and those amounts may correspond to the ranges provided (e.g., 5-200 mg of a compound of Formula I; 1-500 mg of magnesium; and 1-100 mg of iron). A “compound,” “agent,” or “drug” (terms we may interchange) are included in therapeutically effective doses or amounts, however, whenever the dose or amount is sufficient to ameliorate at least one symptom of an existing disorder (by affecting the symptom and/or by affecting a cause of the disorder) or to prevent or reduce the onset or progression of the disorder. Where a compound of Formula I and magnesium are included in the same composition or co-administered, the compound of Formula I and the magnesium may both be present in therapeutically effective amounts. Alternatively, the compound of Formula I may be present in a therapeutically effect amount and the magnesium may be present in an amount sufficient to potentiate the effect of the compound of Formula I.

Topiramate has been reported to cause RLS at doses of 100 mg/day and 200 mg/day (Romigi, et al., J. Neurology, ePub Apr. 30, 2007). Accordingly, one may strive for administration at doses lower than 100 mg/day to prevent adverse affects such as RLS. Inclusion of magnesium and/or iron in the present compositions and methods should facilitate that aim. Generally, inclusion of magnesium and/or iron in the present compositions and methods should allow the active compounds of Formula I to be administered at levels lower than previously suggested.

Suitable doses of a compound of Formula I, particularly when co-administered with magnesium, can be in the range of about 5 mg per dose or per day to about 200 mg per dose or per day (e.g., about 10-100 mg/dose (or 20-200 mg/day); about 15-75 mg/dose (or 30-150 mg/day); about 20-50 mg/dose (or 40-100 mg/day); about 30 mg/dose or 60 mg/day; or about 10-30 (e.g., 25) mg/dose (or 20-60 (e.g., 50) mg/day)). While no more than one to two daily doses is preferable to minimize inconvenience to the patient and improve compliance, additional doses may be prescribed as necessary. Accordingly, the desired dose may be presented in a single dose or as divided doses administered at appropriate intervals, for example, as two three, four or more sub-doses per day. Sustained release formulations, which are known in the art and described further below, can be used to minimize the number of doses required and/or to better maintain an effective dose.

For treating cluster headaches, a composition including a compound of Formula I can be employed at a daily dosage in the range of about 15 to 1000 mg or less (e.g., about 25 mg to about 400 mg; about 25 mg to about 200 mg; or about 10 mg to about 50-100 mg).

In general, suitable doses of magnesium, when co-administered with a compound of Formula I, are in the range of about 1 mg of elemental magnesium per dose or per day to about 500 mg per dose or per day. For example, the pharmaceutical compositions can include elemental magnesium at about 100 mg per dose or per day to about 300 mg per dose or per day (e.g., about 250 mg per dose or per day). Preferably the dose of magnesium given is below the amount which causes toxicity and adverse affects in human adults and human children. The desired dose may be presented in a single dose or as divided doses administered at appropriate intervals, for example, as two, three, four or more sub-doses per day.

Where iron is included in the present compositions and methods, it can be co-administered in the range of about 1 mg/day to about 100 mg elemental iron per dose or per day (e.g., about 5-100; about 10-90; about 20-80; about 40-60 (e.g., 50); about 5-50; or about 10-30 (e.g., 25) mg per dose or per day). Where ranges are provided for any parameter specified herein, it is to be understood that the invention can be practiced with the quantities at or around the endpoints or any value in between. For example, administering about 10-30 mg of iron per dose describes administration of 9, 10, 11, 12, 13, 14, 15 . . . 29, 30, and 31 mg of iron per dose. Preferably the dose of iron co-administered is below the dose considered toxic to human adults and human children and below that dose which causes anaphylactic shock in humans. As with the other active ingredients of the present compositions, the amount of iron required to achieve the desired therapeutic effect may be presented in a single dose or as divided doses administered at appropriate intervals (e.g., as two, three, four or more sub-doses per day). In one alternative, vitamin C is also co-administered when iron is co-administered, in a range of from about 500 mg/day to about 3000 mg/day, in single or divided doses, to improve the absorption of the iron by the subject. In one alternative, multivitamins containing the iron and/or the vitamin C are taken by the subject.

The present compositions including a compound of Formula I and magnesium (and, optionally, iron) can also be used for amelioration of impulse control disorder, tremor, bipolar depression and autism.

The neurological disorders amenable to treatment can have observable physical symptoms and/or behavioral components. For example, the present compositions can be used in the treatment an Impulse Control Disorder (ICD), which includes addiction to food or chemical substances (e.g., alcohol, cocaine, heroin, or phencyclidine (PCP)), tremors, manic-depressive illnesses, and autism. ICDs are characterized by harmful behaviors performed in response to an impulse, drive, or temptation patients find irresistible. For example, the ICD can manifest as intermittent explosive disorder (ED), kleptomania, pathological gambling, pyromania, trichotillomania, compulsive buying or shopping, repetitive self-mutilation, nonparaphilic sexual addictions, severe nail biting, compulsive skin picking, personality disorders with impulsive features, attention deficit/hyperactivity disorder, binge eating, bulimia nervosa, anorexia nervosa with binge eating, or as a substance abuse disorder. Where the present compositions are used to treat an ICD, they can include, or can be administered together with, one or more of a serotonin re-uptake inhibitor, an antidepressant, a psychostimulant, orlistat, and sibutramine.

Tremors, whether familial, essential or senile, result from neurological disorders that produce observable physical symptoms. These tremors are typically in the class of action tremors that oscillate with a frequency of about 4-8 Hz, with variable amplitude. The familial form tends to be inherited as an autosomal trait. While tremors can begin in childhood, onset in adulthood is more typical. If an inheritance pattern is not evident, the tremor is referred to as an essential tremor (also known as benign or idiopathic tremor). These tremors begin in adulthood, while tremors that become evident late in life are known as senile tremors. Information such as this is provided to assist one in identifying a patient amenable to treatment. Diagnostic methods for all of the conditions described herein are, however, well known in the art.

For treating tremors, the present compositions can include a compound of Formula I in the range of about 15 mg to about 500 mg or less (e.g., about 100 mg to about 400 mg or about 25 mg to about 75 mg, administered one to four times per day). A unit dose typically contains about 16 mg to about 300 mg, preferably, about 16 mg to about 200 mg, of the compound of Formula I. With the co-administration of magnesium, the amount of that compound may be effectively reduced.

Another neurological disorder that can be treated with the present compositions is dementia. More particularly, the present compositions can improve or slow the progression of the general impairment of intellectual functioning by, for example, improving or slowing the progression of memory loss, disorientation, misperceptions, difficulties with language, alterations in mood (including undesirable changes in personality and behavior), impaired judgment, and abstraction. The dementia may be associated with Alzheimer's disease; may be vascular dementia; or may be dementia of another type. A patient can be treated with the present compositions whenever they exhibit the primary or cognitive symptoms of dementia (e.g., general impairment of intellectual functioning with behavioral and psychotic disturbances, which may be evident as impaired self care (e.g., an inability to dress, eat, or bathe appropriately), agitation including motor restlessness, verbal and physical aggression, sleep disruption, wandering and incontinence, repetitive behaviors, disinhibition, including inappropriate sexual behaviors, agitation, restlessness, panic, intensified disorientation and verbal or physical outbursts, which can typically occur in the afternoon or evening, and psychotic symptoms such as delusions, hallucinations, and paranoia).

For treating dementia and/or the behavioral and psychotic disturbances that occur in dementia, a compound of Formula I can be administered at a total daily dosage in the range of about 15 mg to about 500 mg or less (e.g., about 15 mg to about 400 mg or less for an average adult human (e.g., about 15 mg up to less than 100 mg), administered one to four times per day). A unit dose can contain about 16 mg to about 300 mg or less (e.g., about 20-80 mg). For treating dementia, in addition to a compound of Formula I, magnesium and, optionally, iron, the present compositions can include, or can be co-administered with, a non-typical antipsychotic such as Risperdal® (risperidone).

Alzheimer's disease is only one of the chronic neurodegenerative conditions that can be treated with the present compositions. Other conditions include Parkinson's disease (in which case patients often experience both the tremors and the dementia described herein), Huntington's disease, amyotrophic lateral sclerosis (ALS), multiple sclerosis, diabetes and diabetic neuropathies (which can be associated with obesity), retinopathy, peripheral nerve injury and brain and spinal neurodegeneration arising as a result of head trauma or spinal injury. In pediatric patients, treatable cases involving brain damage includes those arising from periventricular leukomalacia, cerebral palsy, mental retardation and neonatal stroke. With regard to eye health, in addition to retinopathy, the present compositions can be used to treat macular degeneration and/or optic nerve degeneration.

For treating ALS, a compound of formula (I) has been suggested at a daily dosage in the range of about 100 to 800 mg, usually two divided doses, for an average adult human. A unit dose would contain about 25 to 200 mg of the active ingredient. That dosage or less can be incorporated in the present pharmaceutical compositions together with magnesium or co-administered with magnesium to treat ALS or other chronic neurodegenerative diseases.

Two of the more complicated disorders that can be treated with the present compositions are post-traumatic stress disorder (PTSD) and manic-depressive biopolar disorder (MDBD). PTSD involves multiple neurobiological systems that mediate cognitive, emotional and behavior processes. MDBD is a progressive psychiatric disorder that has been associated with electrophysiologic kindling (Goodwin and Jamison, Manic-Depressive Illness, Oxford University Press, New York, pp 405-407, 1990). In rats, topiramate blocks kindled seizures (Wauquier and Zhou, Epilepsy Res. 24:73-77, 1996). For treating PTSD, a compound of Formula I may be administered at a daily dosage in the range of about 32 to 512 mg or less (e.g., about 30 to 100 mg or less), usually in two divided doses, for an average adult human. A unit dose would contain about 16 to 128 mg of the active ingredient, or less. For treating MDBD, a compound of Formula I may be administered at a daily dosage in the range of about 50 to 200 mg or less (e.g., about 50 to 100 mg or less), usually in two divided doses, for an average adult human.

In addition to depression in the context of MDBD, other types of depression, including treatment-refractory depression, resistant depression, anxious depression and dysthymia can also be treated with the present compositions, which may be administered alone or in combination with a second agent. The second agent can be a mono-amine oxidase inhibitor, a tricyclic compound, a serotonin reuptake inhibitor, a noradrenaline reuptake inhibitor, a dietary supplement, a neuropeptide, a compound that targets neuropeptide receptors, or a hormone. More specifically, the second agent can be: imipramine, amitriptyline, desipramine, nortriptyline, doxepin, protriptyline, trimipramine, maprotiline, amoxapine, trazodone, bupropion, chlomipramine, fluoxetine, citalopram, sertraline, paroxetine, fluvoxamine, nefazadone, venlafaxine, milnacipran, reboxetine, mirtazapine, phenelzine, tranylcypromine, moclobemide, Kava-Kava, St. John's Wart, s-adenosylmethionine, thyrotropin releasing hormone, neurokinin receptor antagonists, triiodothyronine, or a combination thereof. The compound of Formula I can be administered at about 10 to 650 mg daily, or less (e.g., about 16 to 325 mg, or less, once or twice daily). For example, one can administer a pharmaceutical composition that includes about 10 to 100 mg of a compound of Formula I per dose or per day and magnesium (e.g., about 250 mg of elemental magnesium).

Autism is also a complex syndrome that is being increasingly recognized and refers to a group of syndromes known as pervasive developmental disorders. Others include Autistic Syndrome, Rett's Syndrome, Asperger's Syndrome, and Atypical Autism. These disorders are largely behaviorally defined and feature qualitatively impaired social interactions, language and communication difficulties, and a diminished range of interests. As used herein, “autism” covers all such disorders. In a proportion of cases, autism is associated with seizures, which progresses to status epilepticus in slow wave sleep; Beaumanoir and Bureau et al., Eds., Continuous spikes and waves during slow wave sleep-electrical status epilepticus during slow wave sleep-Acquired epileptic aphasia and related conditions. John Libbey, 1995). Autistic regression may also overlap with acquired epileptic aphasia (Landau-Kleffner Syndrome).

To aid in diagnosis, abnormal plasma levels of glutamate have been found in some autistic children (Moreno-Fuenmayor et al., Investigacion Clinica 37:113-28, 1996), and genes for the three GABA_{A} receptor subunits on chromosome 15q have been shown to have aberrations (Schroer et al., Am. J. Med. Genetics 76:327-336, 1998). There are also serotonin abnormalities in autism (Cook and Leventhal, Current Opinion in Pediatrics 8:348-354, 1996), which may be treated through GABA and glutamate alterations induced by compounds of Formula I.

For treating autism, compositions including a compound of Formula I may be employed daily using a pediatric dosage of the compound in the range of about 10 to 200 mg, usually in two divided doses, for an average child no younger than age two. A unit dose would contain about 25 to 200 mg of the compound and would be co-administered with magnesium.

Where the present compositions are administered to alleviate neuropathic pain, including neuralgia, they may include a compound of Formula I in a therapeutically effective amount of the compound (e.g., 10-200 (e.g., about 10-50 to about 50-100) mg per dose or per day). In one embodiment, the compound of Formula I can be included at 50 to 400 mg or less or 25 to 200 mg or less.

In any of the present methods, the compound can be topiramate. The compound of Formula I is co-administered with magnesium, present in a sufficient quantity to have a therapeutic effect or to potentiate the effect of the compound of Formula I.

In treating neuropathic pain, one can further co-administer at least one antagonist for an NMDA receptor or a substance that blocks a major intracellular consequence of NMDA receptor activation. The antagonist or the substance should be non-toxic and either may be pharmaceutically active or may potentiate the effect of the compound of Formula I.

NMDA receptor antagonists include dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), their pharmaceutically acceptable salts, and mixtures thereof. Other useful NMDA receptor antagonists include pyrroloquinoline quinone and cis-4-(phosphonomethyl)-2-piperidinecarboxylic acid. Alternatively, or in addition, the present compositions can include at least one substance that blocks an intracellular event that results from NMDA receptor activation. The major consequences of NMDA receptor activation include:

a) translocation and activation of protein kinases such as protein kinase C, which results in phosphorylation of substrate proteins such as cytosolic enzymes, channel proteins, receptor proteins, and the like;

b) initiation of early gene (e.g., c-fos, c-jun, and zif-268) expression by either increased intracellular Ca²⁺ or Ca²⁺-activated protein kinases, which results in expression of functional genes responsible for, for example, production of cellular enzymes (such as protein kinases), receptor proteins (such as the NMDA receptor), ion channel proteins (such as thos active in K+, Na+, Ca++ channels), and neuropeptides (such as dynorphin); and

c) activation of enzymes (e.g., nitric oxide synthase) and other cellular components by Ca²⁺/calmodulin or other Ca²⁺ binding proteins);

Accordingly, useful substances that may be co-administered with the present compositions with a compound of Formula I and magnesium and/or iron, are substances that interfere with translocation and activation of protein kinase C or with calmodulin induced activation of constitutive nitric oxide synthase as well as induction of inducible nitric oxide synthase. More specifically, the substance can be: an inhibitor of protein kinase C (e.g., a ganglioside such as ganglioside GM₁ or ganglioside GT_{1b}; an amphipathic long chain base (e.g., sphingosine, N,N,N-trimethylsphingosine, sphinganine and psychosine); a quinolyloxazole-2-one (e.g., 4-methyl-5-(3-quinolinyl)-2-(3H)-oxazolone or phenyl-5-(2-quinolinyl)-2-3(3H)-oxazolone); a 1,4-bis-(amino-hydroxyalkylamino)-anthraquinone (e.g., 1,4-bis-(3-propylamino-2-hydroxypropylamino)-9,10 anthracenedione or 1,4-bis-(3-benzylamino-2-hydroxypropylamino)-9,10 anthracenedione; a pharmaceutically acceptable salt of any of the foregoing; or a mixture or any of the foregoing, including substances in salt form.

Additional useful substances include an inhibitor of calmodulin, such as a phenothiazine (e.g., chlorpromazine, chlorpromazine sulfoxide, prochlorperazine dimaleate, perphenazine, trifluoperazine, fluphenazine, fluphenazine enanthate, fluphenazine decanoate, thioridazine, mesoridazine besylate, piperacetazine, acetophenazine dimaleate, carphenazine dimaleate, butaperazine dimaleate and phenothiazine sulfoxide); a naphthalenesulfonamide (e.g., N-(6-aminohexyl)-5-chloro-1-naphthalenesulfonamide, N-(6-aminohexyl)-5-chloro-2-naphthalenesulfonamide or N-(6-aminohexyl)-5-bromo-2-naphthalenesulfonamide); a 4-substituted-4H,6H-pyrrolo[1,2-a][4,1] benzoxazepine (e.g., 1,3-dihydro-1-{1-[(4-methyl-4H,6H-pyrrolo[1,2-a][4,1] benzoxazepin-4-yl)methyl]-4-piperidinyl}-2H-benzimidazol-2-one); a benzhydryl (e.g., N-[2](diphenylmethylthioethyl)-2-(trifluoromethyl)-benzeneethanamine, N-[2-(bis(4-fluorophenyl)methylthio)-)ethyl]-2-(trifluoromethyl)benzeneeth anamine or N-[2-(bis(4-fluorophenyl)methylthio)ethyl]-3-(trifluoromethyl)benzeneethan amine); a tricyclic antidepressant drug (e.g., imipramine, 2-chloroimipramine or amitriptyline); penfluridol; haloperidol; pimozide; clozapine; calmidazolin; a pharmaceutically acceptable salt of any of the foregoing; or a mixture of any of the foregoing, including substances in salt form.

In addition to neuropathic pain, the present compositions can be used to treat abdominal pain, such as that associated with a gastrointestinal disorder such as irritable bowel syndrome (IBS). Thus, the present compositions can also be used in the treatment of conditions that are not usually associated with neurological disorders. Other such disorders include the treatment of benign tumors, cancers, neoplasias, and/or inflammatory disorders or diseases. The benign tumor can be, for example, a hemangioma, hepatocellular adenoma, acoustic neuroma, neurofibroma, lipoma, or a benign bone tumor. The cancer or neoplasia can be primary or secondary and includes carcinomas and sarcomas.

Disorders affecting the skin by thickening and/or inflammation (e.g., psoriasis) can also be treated.

The present compositions and methods can also be administered to reduce weight, reduce weight gain, or to treat obesity. More specifically, for weight reduction, control of weight gain and/or the treatment of obesity, a composition containing a compound of Formula I, magnesium, and/or iron, can be administered. Such compositions can be administered with or may also contain lamotrigene, carbamezepine, felbamate (felbatol), or a combination thereof (e.g., a combination of valproic acid and carbamezepine). Lamotrigene is an anticonvulsant that blocks sodium channels. It has been administered at doses of 100 mg and 200 mg in adjunctive and mono-therapies. Felbamate is also an anticonvulsant approved as an anti-epileptic drug in 1993. Felbamate is an NMDA antagonist but has been observed to affect other receptor types as well. As weight gain and obesity increase a patient's risk of diabetes, treating overweight subjects may also reduce the subjects' risk of becoming diabetic. The present compositions may be administered to patients who have diabetes or who are pre-diabetic. To control weight gain or treat obesity, the present compositions can include, in addition to a compound of Formula I and magnesium, one or more of: orlistat, sibutramine, axokine, dexamphetamine, phentermine, phenylpropanolamine, and/or mazindol. As is true of any of the secondary agents or combination therapies described herein, these agents can be formulated together with a compound of Formula I and magnesium or administered separately.

The present compositions can also be used to lower lipids in a patient, lower blood glucose levels, and/or lower blood pressure. For lowering lipids or blood pressure, a compound of Formula I and magnesium and/or iron may be administered periodically (e.g., daily). The compound of Formula I can be administered in the range of about 100 mg to 400 mg, or less, usually in two daily divided doses, for an average adult human. A unit dose can contain about 15 to 200 mg of the compound of Formula I.

Certain treatments for neuropsychiatric disorders include applying a stimulus (e.g., an electrical or magnetic stimulus) to the patient's brain. The present compositions can be administered before or during the course of such treatments to reduce the risk of, or the duration or severity of, a seizure.

For many of the conditions described above, administration of a compound of Formula I has been suggested previously. Where that is the case, the present methods require administration of not only such a compound, but also magnesium and, optionally, at least one other agent (e.g., iron).

In contrast, it has now been determined that several additional conditions can be treated with a compound of Formula I, and the invention encompasses methods of treating those conditions with either a compound of Formula I without magnesium or by administration of a compound of Formula I together with magnesium. In either case, an additional agent (e.g., iron) can also be co-administered as described herein.

These “additional” conditions include RLS, PLMD, narcolepsy, and more general sleep-related conditions such as fatigue, excessive sleepiness, insomnia, and other disorders characterized by narcoleptic-like symptoms, including Parkinson's Disease.

A subject may experience the symptoms of RLS and/or PLMD on a chronic, daily, occasional, intermittent, sporadic, or infrequent basis. The symptoms characteristic of RLS include, but are not limited to: (1) a desire or urge to move the legs, usually associated with uncomfortable or unpleasant sensations in the legs or other limbs (paresthesias/dysesthesias), (2) motor restlessness, (3) worsening or exclusive presence of symptoms at rest, and (4) worsening of symptoms during the evening or night. The term RLS, as used herein, includes both idiopathic and secondary RLS, which can occur in patients with, for example, uremia, pregnancy, or iron deficiency.

Subjects treated with the featured methods can have refractory RLS, which occurs when a patient has not responded to generally adequate therapy, such as with treatment with a dopamine agonist. Refractory RLS may be characterized by, for example: (1) inadequate initial response despite adequate doses, (2) response that has become inadequate with time, despite increasing doses, (3) intolerable adverse effects, and (4) augmentation that is not controllable with earlier doses of the drug. “Augmentation” is the worsening of RLS symptoms at a particular time, such as early in the day after an evening dose of medication. Augmentation includes earlier onset of symptoms, increased intensity of symptoms, or spread of symptoms to the arms.

In some cases, the subject who has RLS is pregnant, or has end-stage renal disease, or iron deficiency/anemia.

Iron insufficiency may be a feature of RLS. Orally administered iron supplements can sometimes correct iron deficiency and reduce RLS symptoms. In one alternative, iron is added to the therapeutic regimen of co-administering magnesium and a compound of Formula I. In another alternative, vitamin C is included in the regimen described above (the regimen including co-administration of magnesium, a compound of Formula I, and iron) to promote absorbability of the iron. The iron can be supplied as a multivitamin, or can be included in a composition containing magnesium or a compound of Formula I, or both, for use as described above.

PLMS occurs in about 80% of people with RLS, and PLMS is also common in conjunction with certain other disorders and among the elderly. PLMD, also called nocturnal myoclonus, is a sleep disorder where the patient moves limbs involuntarily during sleep and has symptoms or problems related to the movement. Both PLMS and PLMD can cause insomnia, daytime sleepiness, sleep disturbances, narcoleptic-like symptom, and/or narcolepsy. Symptoms of narcolepsy include sleepiness, cataplexy, hypnagogic hallucinations, and sleep paralysis.

The present compositions can also be used to treat fatigue, excessive sleepiness, insomnia, night-awakenings, reduced sleeping, narcoleptic-like symptoms and/or narcolepsy that the subject suffers because the subject has RLS, PLMS, and/or PLMD.

In addition, the present compositions can be used to treat subjects suffering from pre-eclampsia and eclampsia. These conditions are hypertensive diseases of pregnancy for which magnesium sulfate is typically a first-line treatment. Preeclampsia is a medical condition where hypertension arises in pregnancy (pregnancy-induced hypertension) in association with significant protein in the urine, typically diagnosed as proteinuria. Its cause remains unclear. Pre-eclampsia may develop at varying times during pregnancy and its progress differs among patients; most cases are diagnosed pre-term. It has no known cure, but resolves after the patient is no longer pregnant (although it may persist up to about six weeks post-partum).

The present compositions can be used to treat or prevent seizures in a post-partum patient, who has had one, more, or no seizures.

The present compositions can also be administered at any stage to relieve the symptoms of preeclampsia. For example, co-administration can commence upon diagnosis of hypertension or proteinuria, regardless of whether the diagnosis is made pre-term or post-term. Hypertension is typically diagnosed when the subject has two separate readings, taken at least 6 hours apart, of a blood pressure of 140/90 or more. Proteinuria is typically diagnosed when the subject clears 300 mg of protein in 24 hours by urine. Edema (especially in the hands and face) can indicate the onset of preeclampsia.

Eclampsia is a more serious complication of pregnancy and is characterized by convulsions. Usually, eclampsia occurs after the onset of preeclampsia, although preeclamptic symptoms are not always recognizable. The convulsions may appear before, during or after labor. Co-administration of the present compositions (i.e., for the “additional” conditions described here, a compound of Formula I, with or without magnesium) can begin after the first convulsion.

In one typical first-line of therapy, women with preeclampsia or eclampsia can be stabilized temporarily with large doses of magnesium sulfate given intravenously to forestall seizures while steroid injections are administered to promote fetal lung maturation. As provided herein, this line of therapy can be supplemented or supplanted with administration of a compound of Formula I in the absence of magnesium or co-administration of a compound of Formula I, such as topiramate, with magnesium. Typically, the treatment of seizures in eclampsia consists of prevention of convulsion with magnesium sulfate, control of blood pressure with hydralazine, and delivery of the fetus. A co-administration therapy of magnesium and a compound of formula (I) can be given, for example, after delivery of the fetus and maintained until convulsions stop, and blood pressure returns to normal levels. In one embodiment, co-administration therapy of magnesium and a compound of formula (I) is continued for up to two, four, or six weeks, or longer, after delivery of the fetus.

In some embodiments, an additional therapeutic agent can be administered with the magnesium and the compound of Formula I and, optionally, iron. Such agents for treating RLS, PLMS, PLMD, and/or fatigue, excessive sleepiness, and/or narcolepsy associated with these disorders, include, e.g., dopamine agonists, opioids and benzodiazepines. In certain embodiments, the co-administration of magnesium and compound of Formula I can be in addition to the administration of levodopa/carbidopa, carbidopa/benserazide, sustained-release carbidopa/levodopa, pergolide, cabergoline, rotigotine, pramipexole, ropinirole, clonazepam, temazepam, zolpidem, gabapentin, carbamazepine, valproate, bromocriptine, apomorphine, clonidine, baclofen, tramadol, amantadine, estazolam, fluraepam, quazepam, triazolam, alprozolam, clorazepate, chlordiazepoxide, diazepam, lorazepam, ozazapam, eszopiclone, zaleplon, zolpidem, hydrocodone, codeine, fentanyl, hydromorphone, levorphanol, meperidine, morphine, controlled-release morphine, oxycodone, sustained-release oxycodone, pentazocine, propoxyphene, lamotrigine, levetiracetam, oxcarbazepine, pregablin, tiagabine, zonisamide, and methadone. The additional therapeutic can be administered before, at the same time as, or after treatment with magnesium and the compound of Formula I.

In some embodiments, an additional therapeutic agent may be co-administered to treat preeclampsia, eclampsia, and seizures and headaches in a post-partum subject. Such agents include, e.g., hydralazine, labetalol, nitroprusside, phenyloin, diazepam, and other antihypertensives and anticonvulsants. The additional therapeutic can be administered before, at the same time as, or after treatment with magnesium and the compound of Formula I.

In one embodiment, a co-administration therapy of magnesium and a compound of formula (I), such as topiramate, and optionally, iron, can be given as a second-line therapy, such as in the case where a first-line of therapy (such as any of the therapies described above) fails to adequately treat the symptoms of a subject. In some cases, the first-line therapy can be stopped before administration of the present compositions, while in other cases, the present compositions can be given in addition to the first-line therapy.

Kits. The pharmaceutical compositions described herein can be provided in one or more forms of a kit. The user of the kit can be either the subject or another party, such as a relative or healthcare professional. The kit can include one or more of the agents described herein (e.g., magnesium and a compound of Formula I (e.g., topiramate)) in a single or separate formulations. Where iron is included, it may be included within a multivitamin that also includes, for example, vitamin C.

The kits can include one or more containers for packaging and/or administering the compositions. For example, the kits can include a container or containers such as a bag and tubing for intravenous administration, a bottle, vial, syringe, spray pump, ampoule, foil packet, blister pack, and the like, which may be separated by dividers or compartments for the composition(s) and informational material. For example, the kit can include a plurality of containers, each containing a unit dosage form of the compound of Formula I (e.g., topiramate) and magnesium. The container(s) can be airtight and/or waterproof and may be labeled to facilitate use.

The informational material can be descriptive, instructional, marketing or other material that relates to the methods described herein and/or the use of the therapeutic agents for treatment. The informational material can include a description of the composition(s), any warnings deemed necessary (e.g., possible side effects, contraindications, and advice in the event of overdose), instructions for use, and the like. In one embodiment, the informational material can include information about production of the compositions described herein, concentration, date of expiration, batch or production site information, and so forth. The informational material is not limited to any particular form (i.e., it can be printed matter, such as printed text, drawing(s), and/or photograph(s); Braille; computer readable material; a video or audio recording; or a combination of these formats). Other information may also be included. For example, the material can include contact information (e.g., a physical address, email address, website, or telephone number, where a user of the kit can obtain substantive information about the composition and/or its use in the methods described herein). The informational material can be protected by a plastic sleeve or packet or may be provided as a laminated card.

In addition to the therapeutic agent or agents, the composition in the kit can include other ingredients, such as a solvent or buffer, a stabilizer, or a preservative. The agent can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit optionally includes a device suitable for administration of the composition, e.g., a syringe or other suitable delivery device. The device can be provided pre-loaded with one or both of the agents or can be empty, but suitable for loading.

Other embodiments are within the claims.

What is claimed is:

1. A pharmaceutical composition comprising (a) a compound of Formula I or a pharmaceutically acceptable derivative thereof: 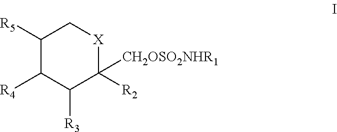
wherein
X is CH₂ or oxygen;
R₁ is hydrogen or lower alkyl; and
R₂, R₃, R₄ and R₅ are independently hydrogen or lower alkyl and R₂ and R₃ and/or R₄ and R₅ together may be a group of the following Formula II: 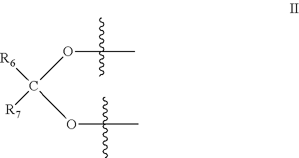
wherein
R₆ and R₇ are the same or different and are hydrogen, lower alkyl or are alkyl and are joined to form a cyclopentyl or cyclohexyl ring, and
(b) magnesium, wherein the amount of the magnesium is sufficient to potentiate the effect of the compound of Formula I or the pharmaceutically acceptable derivative thereof.

2. The pharmaceutical composition of claim 1, wherein X is CH₂ and R₄ and R₅ are alkene groups joined to form a benzene ring.

3. The pharmaceutical composition of claim 1, wherein X is oxygen and R₂ and R₃ and/or R₄ and R₅ together are a methylenedioxy group of Formula II: 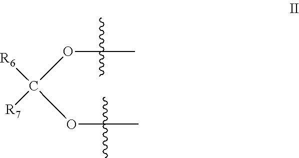
wherein R₆ and R₇ are the same or different and are hydrogen, lower alkyl or are alkyl and are joined to form a cyclopentyl or cyclohexyl ring.

4. The pharmaceutical composition of claim 1, wherein the compound of Formula I is:
2,3-O-(1-methylethylidene)-4,5-O-sulfonyl-beta-D-fructopyranose sulfamate;
2,3-O-(1-methylethylidene)-4,5-O-sulfonyl-beta-L-fructopyranose sulfamate;
2,3-O-(1-methylethylidene)-4,5-O-sulfonyl-beta-D-fructopyranose methylsulfamate;
2,3-O-(1-methylethylidene)-4,5-O-sulfonyl-beta-D-fructopyranose butylsulfamate;
2,3-O-(1-methylethylidene)-4,5-O-sulfonyl-beta-D-fructopyranose ethylsulfamate;
2,3-O-(1-methylethylidene)-4,5-O-sulfonyl-beta-D-fructopyranose octylsulfamate;
2,3-O-(1-methylethylidene)-4,5-O-sulfonyl-beta-D-fructopyranose 2-propenylsulfamate;
2,3-O-(1-methylethylidene)-4,5-O-sulfonyl-beta-D-fructopyranose phenylmethylsulfamate;
2,3-O-(1-methylethylidene)-4,5-O-sulfonyl-beta-D-fructopyranose cyclopropylsulfamate;
2,3-O-(1-methylethylidene)-4,5-O-sulfonyl-beta-D-fructopyranose cyclobutylsulfamate;
2,3-O-(1-methylethylidene)-4,5-O-sulfonyl-beta-D-fructopyranose (2,2,2-trifluoroethyl)sulfamate;
2,3-O-(1-methylethylidene)-4,5-O-sulfonyl-beta-D-fructopyranose dimethylsulfamate;
2,3-O-(1-methylethylidene)-4,5-O-sulfonyl-beta-D-fructopyranose diethylsulfamate;
2,3-O-(1-methylethylidene)-4,5-O-sulfonyl-beta-D-fructopyranose azido sulfamate;
(S)-2,3-O-(1-methylethylidene)-4,5-O-sulfinyl-beta-D-fructopyranose sulfamate;
(R)-2,3-O-(1-methylethylidene)-4,5-O-sulfinyl-beta-D-fructopyranose sulfamate;
2,3-O-(1-ethylpropylidene)-4,5-O-sulfonyl-beta-D-fructopyranose sulfamate;
2,3-O-(1-methylethylidene)-4,5-O—[N-(4-methylbenzenesulfonyl)imidosulfonyl]-beta-D-fructopyranose sulfamate;
2,3-O-(1-methylethylidene)-4,5-O—[N-(4-methylbenzenesulfonyl)imidosulfonyl]-beta-D-fructopyranose sulfamate;
2,3-O-(cyclohexylidene)-4,5-0-sulfonyl-beta-D-fructopyranose sulfamate; or
(S)-4,5-O—[N-(1,1-dimethylethoxycarbonyl)imidosulfinyl]-2,3-O-(1-methylethy lidene)-beta-D-fructopyranose sulfamate.

5. The pharmaceutical composition of claim 1, wherein the compound of Formula I is 2,3:4,5-bis-O-(1-methylethylidene)-beta-D-fructopyranose sulfamate (topiramate).

6. The pharmaceutical composition of claim 1, wherein the pharmaceutically acceptable derivative is a pharmaceutically acceptable salt of a compound of Formula I.

7. The pharmaceutical composition of claim 6, wherein the pharmaceutically acceptable salt is an alkali metal salt, an ammonium salt, or a crystalline choline salt.

8. The pharmaceutical composition of claim 1, wherein the pharmaceutically acceptable derivative is a compound of Formula I that includes a masking chemical group that is dissociated from the compound in vivo.

9. The pharmaceutical composition of claim 8, wherein the chemical group that is dissociated is an imidate group.

10. The pharmaceutical composition of claim 1, wherein the compound of Formula I is present at a unit dosage of 15-75 mg.

11. The pharmaceutical composition of claim 10, wherein the compound of Formula I is present at a unit dosage of 25-50 mg.

12. The pharmaceutical composition of claim 1, wherein the magnesium is present at a unit dosage of 150-400 mg.

13. The pharmaceutical composition of claim 12, wherein the magnesium is present at a unit dosage of about 250 mg.

14. The pharmaceutical composition of claim 1, wherein the composition further comprises iron.

15. The pharmaceutical composition of claim 14, wherein the iron is present at a unit dosage of about 5 to 30 mg elemental iron.

16. The pharmaceutical composition of claim 14, wherein the iron is present in the form of iron (II) sulfate, ferrous gluconate, or NaFeEDTA.

17. The pharmaceutical composition of claim 1, wherein the composition is formulated for oral administration.

18. A method of treating a patient who is suffering from Restless Legs Syndrome (RLS) or Periodic Limb Movement Disorder (PLMD), the method comprising:
(a) identifying a patient in need of treatment; and
(b) administering to the patient a pharmaceutical composition of claim 1.

19. The method of claim 18, wherein the patient has narcolepsy.

20. A method of treating a patient who has, or who is at risk of developing, a condition treatable with topiramate, the method comprising
(a) identifying a patient in need of treatment; and
(b) administering to the patient a pharmaceutical composition of claim 1.

21. The method of claim 20, wherein the condition treatable with topiramate is a neurological disorder.

22. The method of claim 21, wherein the neurological disorder is a condition associated with seizure activity.

23. The method of claim 22, wherein the condition associated with seizure activity is a form of epilepsy or eclampsia or Lennox-Gastaut syndrome.

24. The method of claim 21, wherein the neurological disorder is migraine headache, migraine aura, or cluster headache.

25. The method of claim 21, wherein the neurological disorder is an impulse control disorder.

26. The method of claim 21, wherein the neurological disorder is a chronic neurodegenerative disease.

27. The method of claim 26, wherein the chronic neurodegenerative disease is Alzheimer's disease, vascular dementia, Parkinson's disease, Huntington's disease, multiple sclerosis, amyotrophic lateral sclerosis, or a diabetic neuropathy.

28. The method of claim 21, wherein the neurological disorder is characterized by neuropathic pain.

29. The method of claim 21, wherein the neurological disorder is depression or posttraumatic stress disorder.

30. The method of claim 21, wherein the neurological disorder is the result of head trauma or a spinal injury.

31. The method of claim 20, wherein the patient suffers from bipolar disorder, alcoholism, obesity, optionally associated with binge eating, periventricular leukomalacia, bulimia nervosa, obsessive-compulsive disorder, or idiopathic intracranial hypertension.

32. The method of claim 20, wherein the patient is addicted to nicotine or cocaine.
